(19) United States
(12) Patent Application Publication
(10) Pub. No.: US 2022/0265163 A1
(43) Pub. Date: Aug. 25, 2022

(54) BIOIMPEDANCE SYSTEM FOR ENHANCED POSITIONAL GUIDANCE

(71) Applicants: DATA DRIVEN DIAGNOSTIC SCIENCES, INC., DBA D3SCIENCES, Tucson, AZ (US); ARIZONA BOARD OF REGENTS ON BEHALF OF THE UNIVERSITY OF ARIZONA, Tucson, AZ (US); BANNER UNIVERSITY MEDICAL GROUP, Phoenix, AZ (US)

(72) Inventors: Michael C. Larson, Tucson, AZ (US); Charles T. Hennemeyer, Tucson, AZ (US)

(21) Appl. No.: 17/685,998

(22) Filed: Mar. 3, 2022

Related U.S. Application Data
(63) Continuation-in-part of application No. PCT/US2020/049245, filed on Sep. 3, 2020.
Application No. PCT/US2020/049245 is a continuation-in-part of application No. 16/559,402, filed on Sep. 3, 2019.
Application No. 16/559,402 is a continuation-in-part of application No. 16/559,402, filed on Sep. 3, 2019.
Application No. 16/559,402 is a continuation-in-part of application No. PCT/US18/20851, filed on Mar. 5, 2018.
(60) Provisional application No. 62/466,549, filed on Mar. 3, 2017.

(51) Int. Cl. A61B 5/06; A61B 10/02; A61B 18/14; A61B 34/20; A61B 5/00; A61B 5/0537; A61M 25/01

(52) U.S. Cl. CPC A61B 5/068; A61B 10/0266; A61B 18/1402; A61B 34/20; A61B 5/743; A61B 5/0537; A61M 25/0105; A61B 2018/00601

(57) ABSTRACT

A bioimpedance system is used to obtain multi-bioimpedance measurements for guiding a clinical tool into a body. The system includes a medical instrument that is to be inserted into a body and a plurality of electrodes disposed on a surface of the medical instrument, embedded within the instrument, or both. Each electrode is configured to apply electrical current to the immediate surroundings in contact with the electrode in order to obtain multiple bioimpedance measurements. The bio-impedance measurements are used to guide the medical instrument during insertion and determine positioning and composition of the surrounding environment. The electrodes can also be used to direct application of electricity for cauterizing tissue.

### CROSS REFERENCE

This application is a continuation-in-part and claims benefit of PCT/US2020/049245 filed Sep. 3, 2020, which is a continuation-in-part and claims benefit of U.S. patent application Ser. No. 16/559,402 filed Sep. 3, 2019, the specification(s) of which is/are incorporated herein in their entirety by reference.

This application is also a continuation-in-part and claims benefit of U.S. patent application Ser. No. 16/559,402 filed Sep. 3, 2019, which is a continuation-in-part and claims benefit of PCT/US2018/020851 filed Mar. 5, 2018, which claims benefit of U.S. Provisional Application No. 62/466,549 filed Mar. 3, 2017, the specification(s) of which is/are incorporated herein in their entirety by reference.

### FIELD OF THE INVENTION

The present invention relates to systems and methods for guiding a device into a body using bioimpedance measurements to indicate position of the device and composition of the surrounding environment.

### BACKGROUND OF THE INVENTION

Guidance of instruments, such as catheters, electrosurgical probes, electrosurgical scalpels or knives, scissors, clamps, forceps and needles (hollow in the case of aspiration, partially or semi-hollow in the case of biopsies, or solid as with thermal- or electrical-ablations), is essentially required to maximize efficacy and minimize complications of surgical and percutaneous needle-based procedures. While the current standard of external imaging-based guidance through, for example, x-rays/fluoroscopically/CT, medical ultrasound, or MRI, is very powerful, there are limitations. For instance, CT-guidance for lung biopsies is fraught with difficulties. Patients undergoing lung biopsy are often instructed to hold their breath while a CT scan is performed to determine if the trajectory of the needle is in line with the target lesion. However, many patients cannot reasonably hold their breath for the duration of the scan, let alone the brief time for interpretation of the scan including triangulating the trajectory of the needle and the target and then the time it takes for appropriate advancement of the needle to the target. During this interval from scan to needle advancement to the target, the needle tip moves with respirations and lung movement. Furthermore, despite the most proficient percutaneous intervention, complications related to bleeding do occur from the needle violating blood vessels. As another example, ultrasound (US) has also been used in biopsies. However, US takes images of slices, e.g. 2-dimensional views, therefore performing a biopsy can be a long, interactive process. For laparoscopic surgical procedures, the operator is limited in evaluating organs and tissue to visual appearance only. This may be problematic when different tissues or organs appear very similar. Hence, safer and better guidance and positioning of instruments is desired.

### SUMMARY OF THE INVENTION

According to some embodiments, the present invention features a bioimpedance system for guiding an instrument, such as a needle, inside a body and providing positioning information. The system may comprise an instrument for insertion into the body, one or more electrical connections operatively coupled to the instrument, an impedance spectrometer operatively coupled to the one or more electrical connections, and a processor operatively coupled to the impedance spectrometer. The one or more electrical connections may be wired to the instrument, disposed on a surface of the instrument, or embedded therein. The impedance spectrometer is configured to pass electrical current to the one or more electrical connections. The processor is configured to execute computer-readable instructions that cause the processor to perform operations comprising obtaining bioimpedance measurements from the one or more electrical connections, and determining a composition of a local environment surrounding the instrument, thereby providing guidance during insertion and positioning of the instrument within the body.

In some embodiments, the electrical connections may comprise about 2-128 electrodes that are electrically capable yet isolatable from the other electrodes. In one embodiment, the electrodes may comprise conductive strips, ribbons, or wires disposed axially along the surface of the instrument or embedded within the instrument. In another embodiment, the electrodes may comprise multiple concentric telescoping tubes each with an electrically active exposed tip or surface. In yet another embodiment, the strips, ribbons, or wires disposed axially may be in combination with concentric tubes. In some embodiments, an insulating material may be partially covering the electrodes, a portion of the instrument, or both.

According to some embodiments, the present invention provides a method of guiding insertion and positioning of an instrument inside a body. The method may comprise providing a bio-impedance guided system, inserting the instrument into the body, obtaining bioimpedance measurements using the one or more electrical connections, and determining a position or direction of the instrument based on the multiple bioimpedance measurements.

One of the unique and inventive features of the present invention is the multiple electrodes operatively coupled to the medical instrument. This enables more than one impedance measurement to be obtained for providing spatial information. Without wishing to limit the invention to a particular theory or mechanism, this feature can allow for guided measurements or insertion of the medical instrument using directional information from multiple bio-impedance readings by the electrodes, thereby further reducing the likelihood of intervening off-target and associated complications. None of the presently known prior references or works has these unique inventive technical features of the present invention.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE DRAWINGS

The features and advantages of the present invention will become apparent from a consideration of the following detailed description presented in connection with the accompanying drawings in which:

FIG. 1 shows a non-limiting general schematic of a bioimpedance guidance system in accordance with the present invention.

FIG. 2 is a non-limiting schematic of an electrosurgical bioimpedance guidance system in accordance with the present invention.

FIG. 3 shows a non-limiting embodiment of the electrosurgical bioimpedance guidance system having an electrosurgical probe or knife with integrated electrodes.

FIG. 4 is a non-limiting schematic of a catheter-based wire bioimpedance guidance system in accordance with the present invention.

FIG. 5 shows one embodiment of the catheter-based wire bioimpedance guidance system.

FIG. 6 is a non-limiting schematic of a bioimpedance-guided cryoablation needle system in accordance with the present invention.

FIG. 7 is a non-limiting schematic of a bioimpedance-guided microwave needle ablation system in accordance with the present invention.

FIG. 8 is a non-limiting schematic of a bioimpedance-guided radiofrequency ablation (RFA) system in accordance with the present invention.

FIG. 9 shows a non-limiting embodiment of the bioimpedance-guided RFA system.

FIG. 10 is a non-limiting schematic of a bioimpedance-guided radiofrequency ablation (RFA) system in accordance with the present invention.

FIG. 11 is a non-limiting schematic of a bioimpedance-guided biopsy and electrocoagulation system in accordance with the present invention.

FIG. 12A is a cross-sectional view of a biopsy needle with three electrodes and dielectric material around the inner needle/electrode. Directional information can be obtained by isolating or summing the various electrodes relative to others.

FIG. 12B shows a biopsy needle implementing bioimpedance to provide positional guidance of the needle complementary to external imaging guidance.

FIG. 12C shows the use of bioimpedance feedback to locate where the needle is in contact with blood and where to deliver electrocautery/electrocoagulation to address bleeding.

FIG. 13 shows a non-limiting example of an experimental setup for measuring bioimpedance, featuring an impedance spectrometer with electrode needle and grape, representing a tumor phantom, embedded in a sponge, representing tissue. Needle electrode trajectories are indicated in the right figure.

FIGS. 14A-14E show relative impedance measurements over time through controls, the tissue phantom, and the tumor phantom. In FIG. 4A, the needle electrode in the air has a high impedance control (note the magnitude/vertical axis is generally in the hundreds of thousands to nearly a million ohms). In FIG. 14B, the needle electrode moving through the sponge (serving as a lung tissue phantom) showed alternating areas of relatively high and low bioimpedance. FIG. 14C shows the needle electrode tip in the grape serving as relatively low impedance (representing the tumor phantom; note the impedance is only tens of thousands of ohms). In FIG. 14D, the needle tip moving through-and-through the tumor phantom demonstrates a relatively abrupt drop in impedance upon entry and then increased impedance upon exit. FIG. 14E demonstrates successful placement of the electrode needle tip within the tumor phantom.

FIG. 15A shows an embodiment of a biopsy needle with an outer sheath/cannula acting as a single electrode, and the inner needle as the second electrode. Front and back details have been omitted for simplicity.

FIGS. 15B-15E show non-limiting embodiments of the outer sheath with multiple electrodes.

FIGS. 16A-16E are cross-sectional views showing non-limiting embodiments of the biopsy needle with multiple electrodes and dielectric material around the inner needle/electrode.

FIGS. 17A-17D are cross-sectional views of other non-limiting embodiments of the biopsy needle with multiple electrodes and dielectric material around the inner needle/electrode.

FIGS. 18A-18D are cross-sectional views of various non-limiting embodiments of the biopsy needle with multiple electrodes and dielectric material around the inner needle/electrode.

FIGS. 19A-19F are cross-sectional views of alternative embodiments of the needle with multiple electrodes embedded in the semi-solid or solid needle.

FIG. 20A shows a non-limiting embodiment of a biopsy needle system capable of electrocoagulation during the biopsy procedure.

FIG. 20B shows an outer needle sheath sheathing a deployable cutting mechanism of a biopsy needle. Dashed lines represent objects hidden from view.

FIG. 20C shows the outer needle sheath is a retracted position to deploy the cutting mechanism of the biopsy needle.

FIG. 20D illustrates a biopsy procedure where rotation of the needle results in cutting of tissue and the cut tissue is suctioned into a collection chamber.

FIG. 21A shows a schematic of another non-limiting embodiment of the biopsy needle system.

FIG. 21B shows the outer needle sheath with gear and motor drive over the needle to retract the sheath. A Luer lock can connect a distal sheath to a proximal sheath.

FIGS. 22A-22C show yet another non-limiting embodiment of the biopsy needle system.

FIGS. 23A-23B show the internal components and mechanism of the biopsy needle system shown in FIGS. 22A-22C.

### DETAILED DESCRIPTION OF THE INVENTION

100 bioimpedance system
105 body
110 instrument (e.g. knife, needle, catheter, probe, forceps, clamps, scissors)
120 impedance spectrometer
130 computer/processor
162 dielectric/insulating component
164 electrode
200 biopsy system
201 needle
203 needle tip
205 sheath
210 handle
215 vacuum collection chamber
217 collection chamber cap
220 translation spring
225 rotational screw
230 trigger
235 activation button
237 electronic components
240 internal syringe
245 vacuum spring

As known to one of ordinary skill, bioimpedance is the measurement of resistance to alternating current flow in a biological medium, organism, or specimen. Resistance depends on the resistivity of different mediums to the electrical current. For example, body fluids are similar to electrolytic solutions and make for better conductors than bone and fatty tissues. Bio-impedance can be used to provide information to a healthcare provider such as, for example, placement of a needle in the desired tissue given that the electrical conductivity of different tissue types is variable.

As known to one of ordinary skill it the art, cauterization involves burning or singeing a target tissue typically to coagulate and stop bleeding and reduce or prevent infections. The cauterized area then heals. Electrocoagulation in a biomedical sense, on the other hand, is the use of electricity to precipitate soluble proteins, as what happens with the blood coagulation cascade.

As used herein, the term “electrocautery/electrocoagulation” refers to the use of electricity through cauterization or coagulation, preferably without significant tissue damage. In some embodiments, electrocautery/electrocoagulation applies high frequency alternating current by a unipolar or bipolar method. The high frequency alternating current may be applied intermittently to coagulate tissue.

As used herein, the term “electrosurgery” refers to pulsating at higher frequencies to cut with little thermal damage. The high frequency alternating current may be applied in a continuous waveform to cut tissue. Medical instruments may be operatively connected to an electrical generator or power source for cauterization and/or surgical cutting.

In one embodiment, an example of the electrosurgical and/or electrocautery/electrocoagulation unit that may be used in accordance with the present invention includes a unipolar unit with one polarity on or near the cutting element and a second polarity placed on the patient using an electrode pad and connected to the electrical unit/generator. In another embodiment, the electrosurgical and/or electrocautery/electrocoagulation may comprise a bipolar unit with one polarity on or near the cutting element and a second polarity placed on another part of the instrument within the patient. In yet another embodiment, the conductive clinical tool (e.g. needle, catheters, probes, knives, scissors, clamps, or forceps) may comprise a monopolar unit with one polarity and a second polarity placed elsewhere on the patient, or comprise a bipolar unit incorporating two or more electrodes within the same tool. Such tools may provide electrical bioimpedance data to the operator.

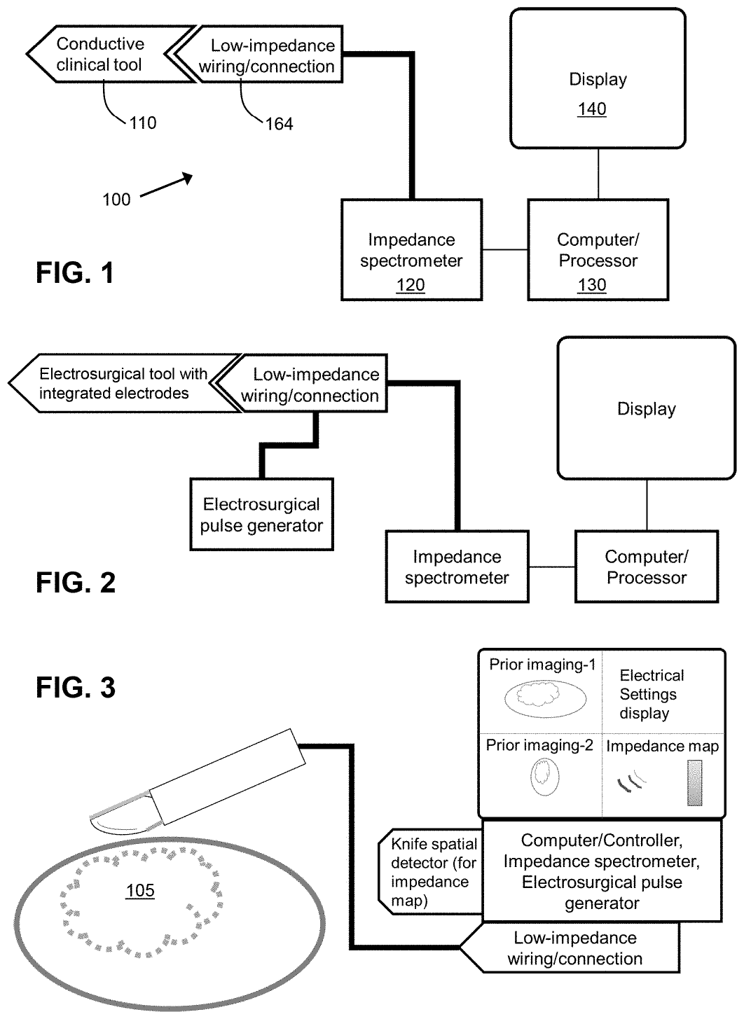

Referring now to the figures, in one embodiment, the present invention features a bioimpedance system (100) for guided positioning of an instrument inside a body (105). In one embodiment, as shown in FIG. 1, the system (100) may comprise an instrument (110) for insertion into the body (105), one or more electrical connections (164) operatively coupled to the instrument, an impedance spectrometer (120) operatively coupled to the one or more electrical connections (164), and a processor (130) operatively coupled to the impedance spectrometer (120). In some embodiments, the one or more electrical connections (164) may be wired to the instrument (110), disposed on a surface of the instrument, or embedded therein. The impedance spectrometer (120) is configured to pass electrical current to the one or more electrical connections (164). The processor (130) is configured to execute computer-readable instructions that cause the processor (130) to perform operations comprising obtaining bioimpedance measurements from the one or more electrical connections (164), and determining a composition of a local environment surrounding the instrument (110), thereby providing guidance during insertion and positioning of the instrument (110) within the body (105). Without wishing to limit the present invention to a particular theory or mechanism, when the instrument (110) is inserted into the body (105), the bioimpedance measurements can indicate bone, tissue type, and/or fluids in the local environment surrounding the instrument (110).

In some embodiments, the electrical connections (164) comprise electrodes. The number of electrodes may be about 3-128 electrodes. In some embodiments, each electrode is electrically capable and can be isolated from the other electrodes. In further embodiments, an insulating material may be partially covering the electrodes, a portion of the instrument, or both. In some embodiments, the plurality of electrodes (164) comprises conductive strips, ribbons, or wires disposed axially along the surface of the outer sheath, the needle surface, or embedded and fixed within the instrument. In other embodiments, the plurality of electrodes (164) comprises multiple concentric telescoping tubes each with an electrically active exposed tip or surface. In some other embodiments, the strips, ribbons, or wires may be used in combination with concentric tubes.

In one embodiment, the instrument (110) may be an electrically conductive clinical tool. Non-limiting examples of the instrument (110) include needles, catheters, probes, surgical tools, knives, scissors, clamps, and forceps. In some embodiments, the instrument (110) may function as an additional electrical connection, e.g. electrode. In other embodiments, the instrument (110) may be comprised of conductive and insulated portions in which the conductive portions function as additional electrical connections. With any of these configurations, the directional information of the instrument can be obtained by isolating or summing the various electrical connections relative to other electrical connections.

In some embodiments, bioimpedance can be implemented with a biopsy needle, but it is not limited to biopsy needles and procedures. For instance, in other embodiments, bio-impedance may be used with needles in an ablation procedure. As known to one of ordinary skill in the art, ablation is a procedure involving the application of energy to destroy tissue. Thus, without deviating from the scope of the present invention, bio-impedance may be used with any medical/clinical instrument or procedure in which guidance knowledge of the instrument's relative position is desired.

Referring now to FIG. 2, in some embodiments, the instrument (110) is an electrosurgical tool. The system may further comprise an electrosurgical pulse generator operatively coupled to the electrosurgical tool. As shown in FIG. 3, the electrosurgical tool may be a scalpel or knife. An electrosurgical knife with integrated electrodes could provide a real-time bioimpedance map when cutting into tissue, supplementing the surgeon's sense of sight and touch when cutting out a mass with different impedance than adjacent tissue. Additionally, bioimpedance could serve as feedback to modulate electrosurgical pulse current to minimize the electrical power dissipated by the surgical patient while still maintaining hemostasis.

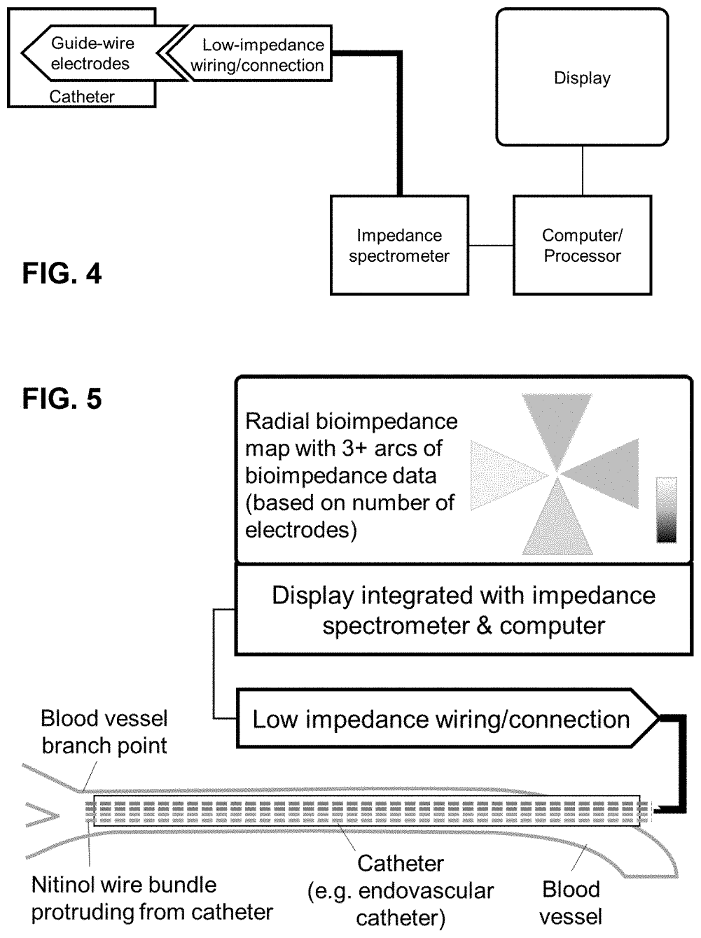

Referring to FIG. 4, in other embodiments, the instrument (110) is a catheter or scope. The electrical connections may comprise a plurality of guide-wire electrodes disposed lengthwise inside the catheter. As a non-limiting example, shown in FIG. 5, the catheter-based wire bioimpedance guidance system may have a Nitinol wire bundle protruding from a catheter. Nitinol, also known as “muscle wire,” could flex in response to electrical changes in bioimpedance to aid in directing wires into different blood vessel branch points, in addition to displaying a radial bioimpedance view. In other embodiments, the bioimpedance system may be used to deliver a scope to a target location in the body.

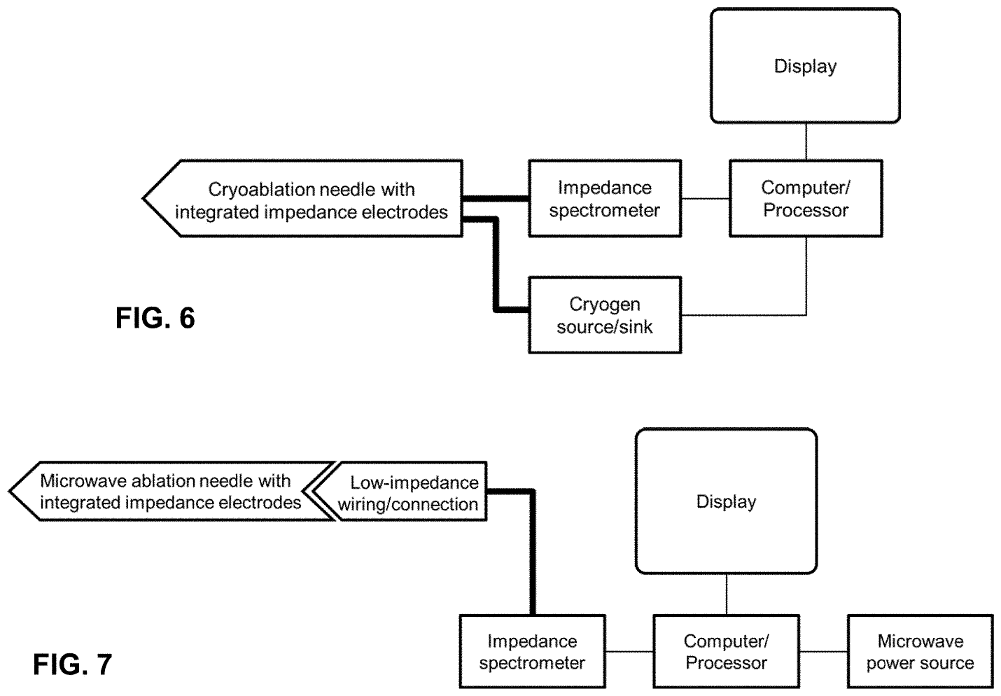

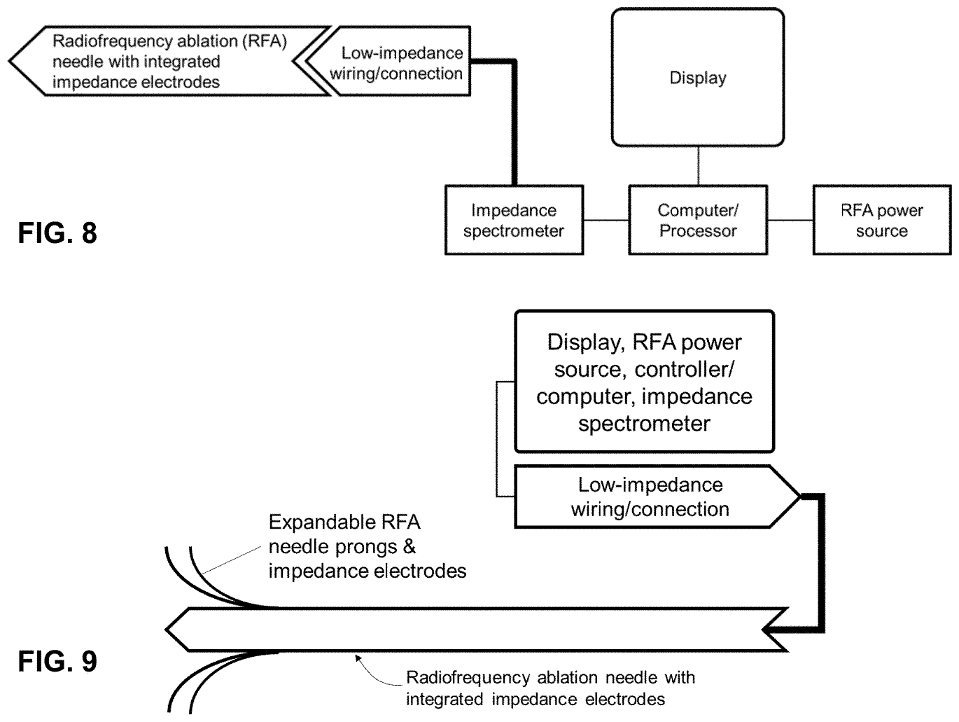

Referring to FIGS. 6-8, in some other embodiments, the instrument (110) may be a cryoablation needle, a microwave ablation needle, or a radiofrequency ablation (RFA) needle. As shown in FIG. 9, in some embodiments, the RFA needle may include expandable RFA needle prongs and impedance electrodes. In one embodiment, the prongs are heat-ablating prongs. With microwave or radiofrequency ablation, there is no easy way to image the ablation cavity size. Heating results in bubble formation, which obscures evaluation with ultrasound, and CT does not have good enough tissue resolution. Impedance is influenced by heat, and may serve as a method to measure the ablation cavity size.

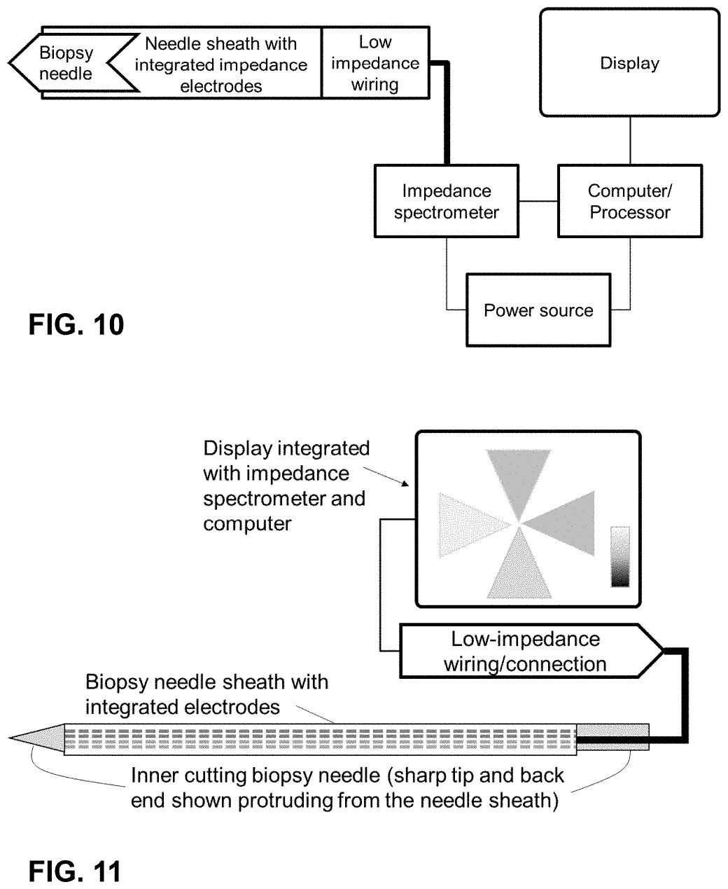

Referring to FIG. 10, the instrument (110) may be a biopsy needle having a needle sheath. The electrical connections comprise electrodes disposed on a surface of the needle sheath. For example, in FIG. 11, the bioimpedance guidance system may include a biopsy needle having a needle sheath with integrated impedance electrodes. Impedance-based guidance could supplement external imaging-based guidance (e.g. ultrasound, CT, fluoroscopy) of the biopsy needle into the tissue or lesion of interest, again using a (radial or 3D spatial) bioimpedance map. When incorporated with electrocoagulation capabilities, bioimpedance could direct where to infuse electricity to stop biopsy-related bleeds. Furthermore, in some embodiments, the instrument (110) may have cauterization capabilities.

In further embodiments, the system (100) may include a memory (not shown) operatively connected to the processor (130). The memory is configured to store the computer readable instructions for execution by the processor. The memory may be any non-transient medium known in the art. In yet other embodiments, the processor (130) may be operatively coupled to a display (140). As demonstrated in FIG. 3, the display can show information including, but not limited to, impedance data, electrical settings, and impedance maps.

In some embodiments, the processor is a computer or controller. In other embodiments, the processor and impedance spectrometer may be integrated into one device. In some other embodiments, the processor, impedance spectrometer, and display may be combined in one device. In yet other embodiments, the processor, impedance spectrometer, electrical pulse generator, and display may be integrated into one device.

The bioimpedance guided systems (100) described herein may be used to improve guidance when positioning the instrument. Accordingly, the present invention provides a method of guiding insertion and positioning of an instrument (110) inside a body (105). In some embodiments, the method may comprise providing a bio-impedance guided system (100) as described herein, inserting the instrument (110) into the body (105), obtaining bio-impedance measurements using the one or more electrical connections (164), and determining a position or direction of the instrument (110) based on the multiple bioimpedance measurements. Without wishing to limit the present invention to a particular theory or mechanism, the bioimpedance measurements provide 360° spherical awareness of the surrounding environment to indicate the position of the instrument and composition of the tissue. For example, the bioimpedance measurements can indicate bone, tissue type, and/or fluids in a bodily environment surrounding the instrument (110). In other embodiments, the method may further comprise advancing the instrument (110) inside the body, changing direction of movement of the instrument, retracting the instrument, and/or ceasing movement of the instrument based on the bioimpedance measurements.

Referring to FIGS. 10, 11, and 20A-21B, a non-limiting embodiment of the bio-impedance guided system (100) may comprise a biopsy system (100) for harvesting a target tissue. The biopsy system (100) may comprise a needle having a tip for insertion into tissue, a lumen disposed in the needle, and an aperture disposed at or near the tip of the needle and fluidly connected to the lumen. The needle includes a cutting mechanism adapted to cut tissue. The cutting mechanism can have at least a portion thereof disposed in or over the aperture. A sheath may be slidably disposed around an exterior surface of the needle. The sheath is adapted to move between at least an open position where the aperture and cutting mechanism are exposed, and a closed position where the aperture and cutting mechanism are covered by the sheath. The biopsy system further comprises a mechanism for cauterizing tissue that contacts said cauterizing mechanism, a mechanism for rotating the needle, one or more electrical connections operatively coupled to the needle, an impedance spectrometer operatively coupled to the one or more electrical connections to pass electrical current to the one or more electrical connections, and a processor operatively coupled to the impedance spectrometer. The electrical connections may be configured to apply electrical current for cauterizing tissue, coagulating blood, obtaining the multiple bioimpedance measurements to guide needle insertion and positioning, and/or initiating electron-dependent biochemical processes. The processor may be configured to execute computer-readable instructions that cause the processor to perform operations comprising obtaining bioimpedance measurements from the one or more electrical connections, and determining a composition of a local environment surrounding the needle, thereby providing guidance during insertion and positioning of the needle within the body;

Without wishing to limit the present invention, when the needle is inserted into a body, the bio-impedance measurements are used to guide and position the needle at the target tissue. Preferably, the needle tip is guided and positioned at a periphery of the target tissue. The sheath is then deployed in the open position and the needle is rotated via the rotation mechanism and simultaneously retracted from the periphery. The cutting mechanism cuts the tissue and directs said cut tissue into the aperture and further into the lumen, while contacting tissue is cauterized by the cauterizing mechanism. In one embodiment, the cutting mechanism comprises a dome-shape structure having a leading cutting edge projecting from the needle. In another embodiment, the cutting mechanism is deployable. Non-limiting examples of the deployable cutting mechanism include at least one cylindrical or filament wire or an expandable dome-shape structure, at least one flat wire with a first side and a second side, wherein the first side is for cutting and the second side is for cauterization or coagulation, or a nitinol memory wire that is pre-configured to assume a desired conformation.

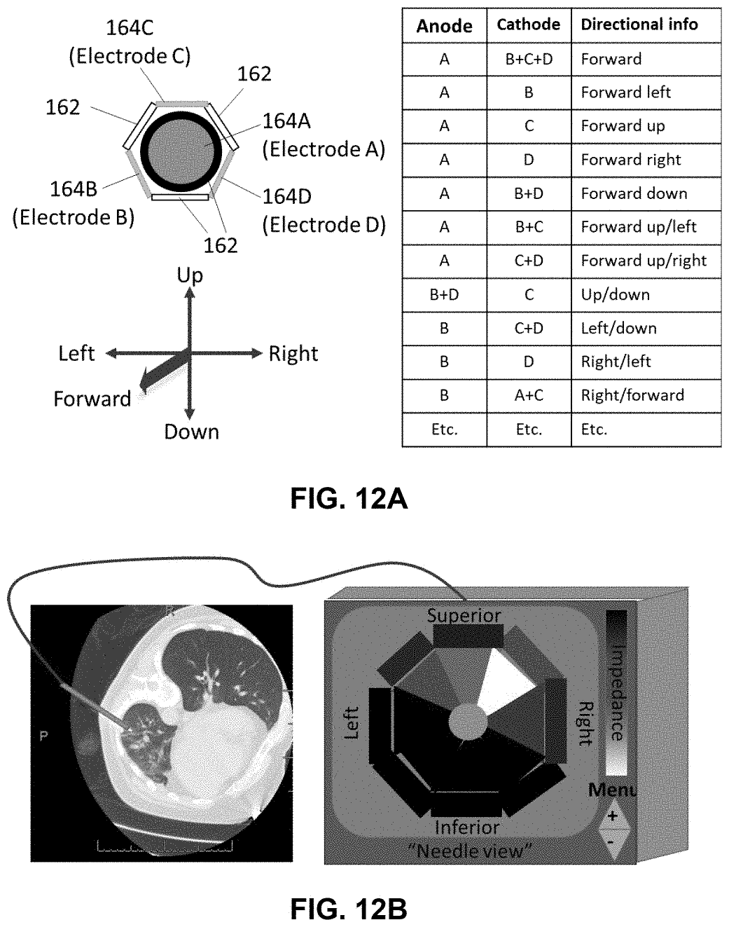

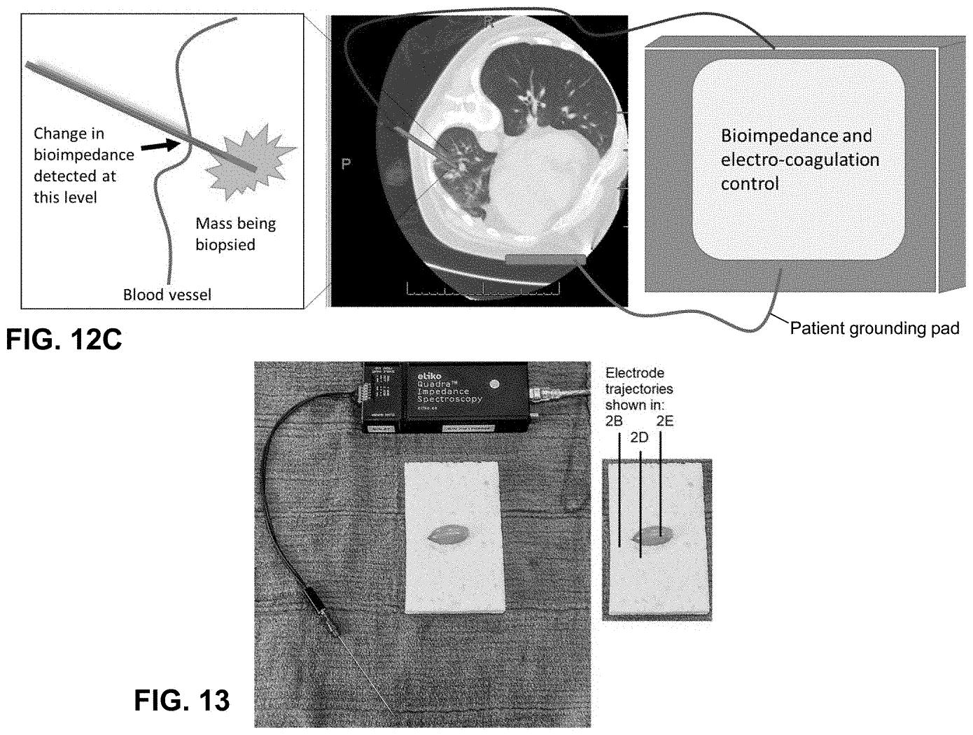

According to some embodiments, the electrical connections may comprise one or multiple electrodes incorporated into an outer aspect, such as the sheath/cannula, of a percutaneous needle device or on the percutaneous needle itself. The geometry of these electrodes determine the spatial information provided for guidance of needle-based, percutaneous procedures. As shown in FIGS. 12A-12C, the biopsy system makes use of both the spatial information provided by the needle and also the relative low bioimpedance of electrolyte-rich blood as a means to direct current flow and resultant electrical coagulation in case of a bleeding complication.

Accordingly, in some embodiments, the present invention provides a method of guiding insertion of a needle into a subject. The subject may be a human or other mammal such as a dog, cat, horse, etc. For example, the subject may be a medical or veterinary patient. In one embodiment, the method may comprise providing a biopsy system (100) as described herein, obtaining multiple bio-impedance measurements from the plurality of electrodes (164), and determining directional information and/or position of the needle based on the multiple bioimpedance measurements.

FIG. 12A shows a non-limiting embodiment of a needle, in a cross-sectional view, with three electrodes and dielectric material around the inner needle/electrode. Referring to the table, in some embodiments, the resultant directional information can be obtained by isolating or summing the various electrodes relative to other electrodes. In preferred embodiments, the present invention may be used to provide positional guidance to a display box, as shown in FIG. 12B. In some embodiments, the bioimpedance directionality may complement in real-time the snap-shots of information obtained during successive external image guidance (e.g. CT) scans to guide the needle.

In some embodiments, the bioimpedance system can be used to provide real-time bioimpedance feedback on where the needle is in contact with blood and where to deliver electrocautery/electrocoagulation to address bleeding. As shown in FIG. 12C, the left-most image is a close-up view of the needle damaging a blood vessel during a CT-guided lung biopsy as demonstrated in the middle image. A change in impedance is detected between blood and tissue. External grounding (optional) to the control and display box can improve the bioimpedance signal-noise and provide more focused electrocoagulation of the damaged blood vessel to prevent bleeding complications.

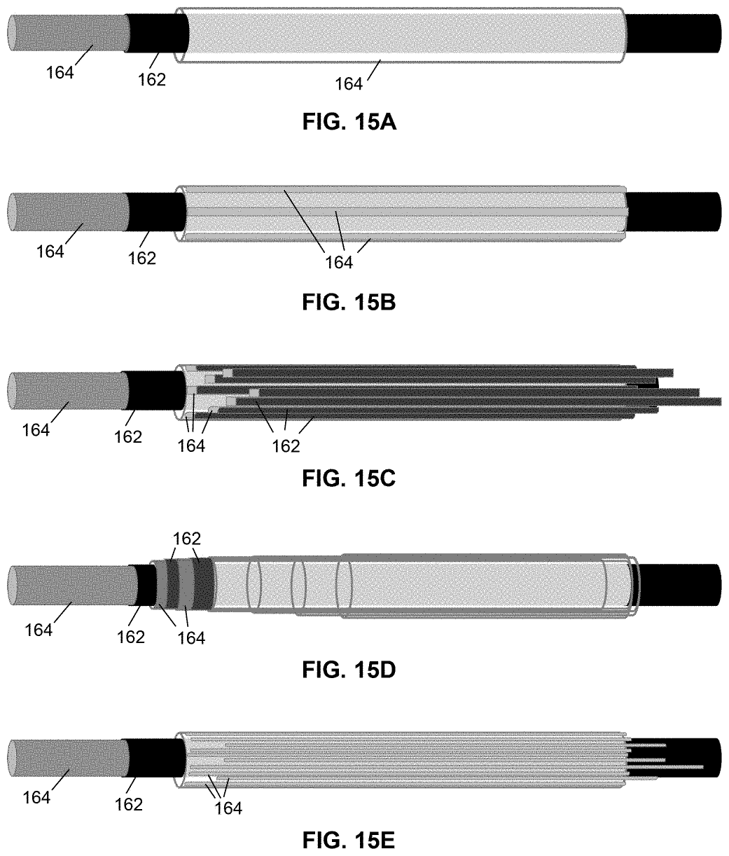

FIG. 15A shows an embodiment of a needle that may be used for electrocautery/electrocoagulation, but does not provide for bio-impedance guided insertion. In contrast, FIGS. 15B-15E show various embodiments of the needle having multiple electrodes that can provide bio-impedance guided insertion. These embodiments are examples of the electrodes placed on the outer sheath. The exposed inner needle can also act as another electrode whereas the black portion is coated with a dielectric or insulating material. In FIG. 15C, the needle can have overlapping, partially-insulated plates making up the outer sheath/guide needle and serving as multiple additional electrodes to guide placement and/or where electricity should be deposited for electrocoagulation. FIG. 15D shows another non-limiting embodiment of multiple concentric overlapping cores with electrically active exposed tips as multiple additional electrodes to guide where electricity should be deposited for safer electrocoagulation. FIG. 15E is yet another embodiment of a wire-enclosed guide needle, with multiple wires serving as additional electrodes. The front and back end connection details have been omitted for simplicity. These electrode configurations are not limited to just needles, and may be applied to other instruments such as scalpels and catheters. For example, electrodes may be placed on the surface of a scalpel. As another example, electrode wires may be disposed inside a cannula.

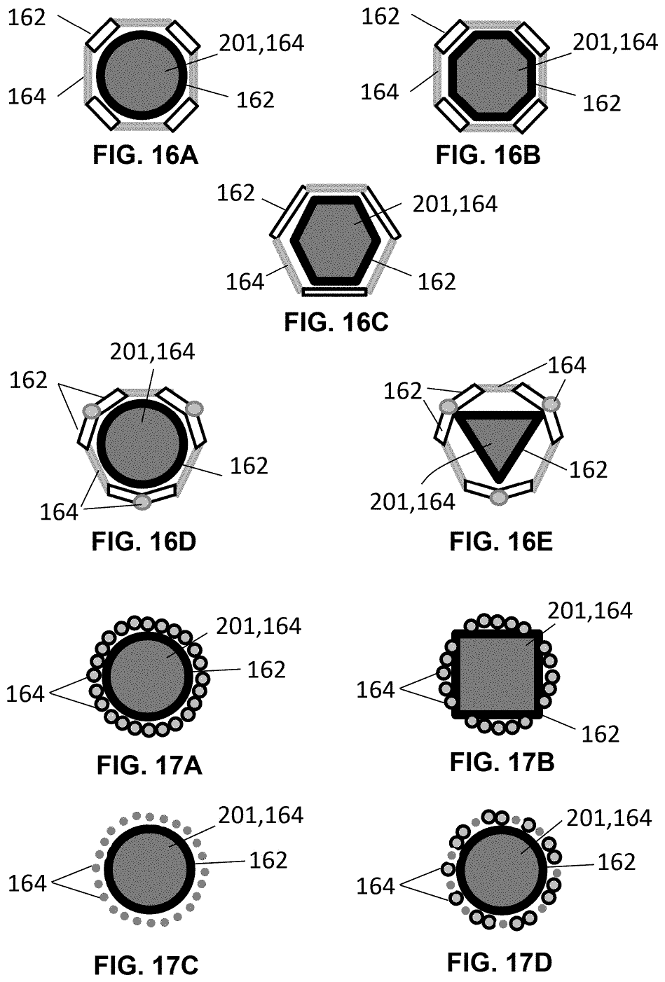

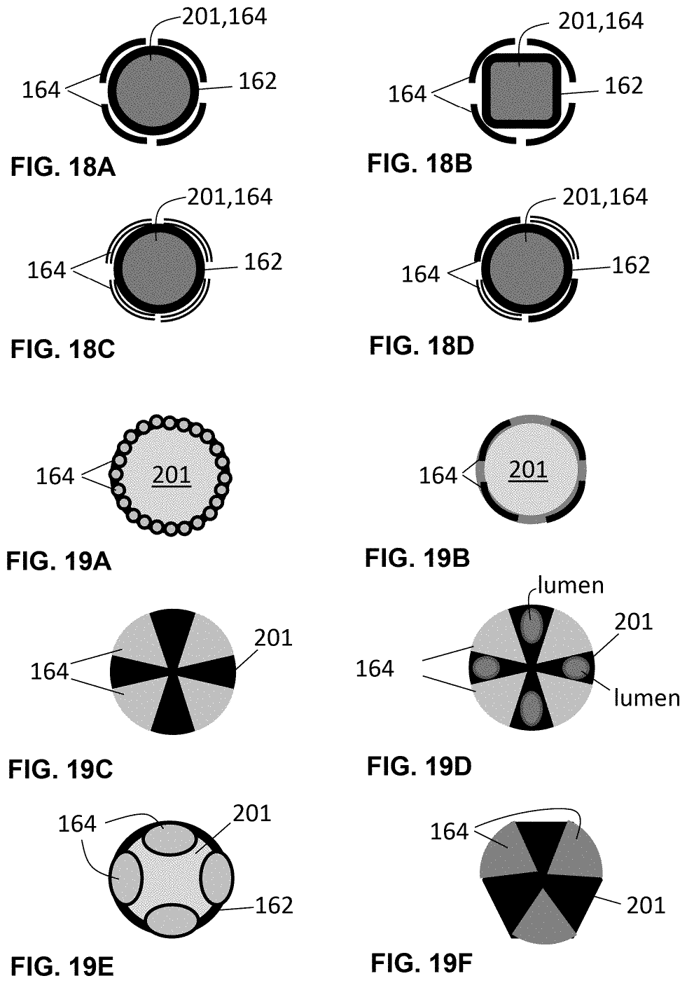

FIGS. 16-16E show alternative embodiments of the arrangement of the electrodes around an instrument, such as a needle. Of note, the needle may be entirely hollow, but it may also be partially hollow or completely solid depending on the clinical application (e.g. aspiration, biopsy, ablation, etc.). In some embodiments, the electrodes can have varying dielectric insulation along the shaft of the needle or the outer sheath. As shown in FIGS. 17A-17D, alternative embodiments of the instrument may include wires that can each serve as an electrode with varying degrees of electrical exposure towards the tip. In some embodiments, the wires may be fully coated, partially coated, or completely uncoated. Referring to FIGS. 18A-18D, in some embodiments, the instrument may have ribbons disposed on the outer sheath. These ribbons can serve as electrodes with varying degrees of electrical exposure towards the tip. In some embodiments, the ribbons may be fully coated, partially coated, or completely uncoated.

FIGS. 19A-19B show alternative embodiments of the instrument where non-removable electrodes are incorporated into the outer surface of the instrument. In these embodiments, the instrument may have an outer sheath, or alternatively, no outer sheath. If the instrument is a needle with an outer sheath, the outer sheath may have electrodes, or alternatively, no electrodes disposed thereon. In some embodiments, the inner needle may be entirely hollow, but it may also be partially hollow or completely solid. For example, the inner needle may have a lumen or no lumen depending on the specific clinical use (e.g. thermal cooling after ablation, aspiration, etc.). FIGS. 19C-19F show non-limiting embodiments incorporating the conductive surfaces into the instrument. For instance, multiple isolated but conductive faces can be incorporated into a solid instrument. The solid instrument may have lumens (e.g. for coolant for thermal applications, for aspiration/injection, etc.) or no lumen. Various embodiments of the instrument may feature a circular cross-section, or a non-circular or non-rounded cross-section. As shown in the figures, non-limiting examples of the cross-section include a square or rectangle, a triangle, or a polygon.

In some embodiments, a plurality of electrodes may be disposed axially on the surface of the instrument. In alternative embodiments, the plurality of electrodes may be disposed radially, e.g. concentric, on the surface of the instrument. In preferred embodiments, the needle may have two or more electrodes. Without wishing to be bound to a particular theory, the plurality of electrodes can provide better or more accurate directional information. With any of these configurations, directional information of the instrument can be obtained by isolating or summing the various electrodes relative to other electrodes.

Although multiple electrodes can be placed within the instrument or on the surface of the instrument or sheath, the overall diameter remains small, thereby reducing pain when the instrument is inserted into a patient. For instance, a maximum thickness of the instrument may be less than 5 mm. In some preferred embodiments, a maximum thickness of the instrument is less than 2 mm. In other preferred embodiments, a maximum thickness of the instrument is less than 1 mm.

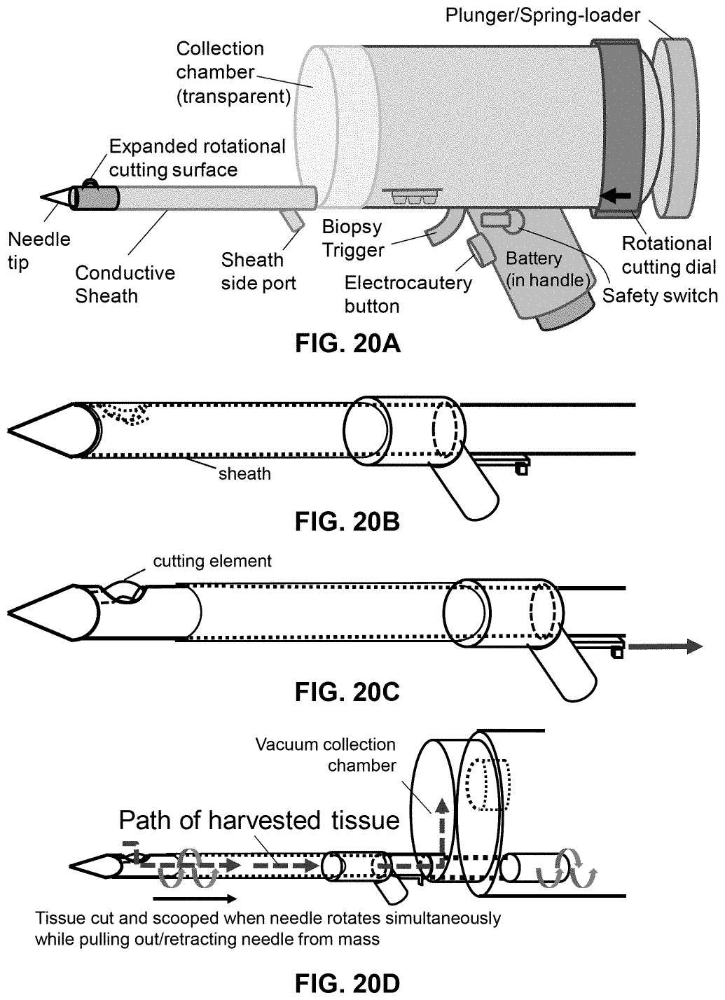

In further embodiments, as shown in FIG. 20A, the biopsy system includes a collection chamber for storing the suctioned tissue. An airtight seal affixes the vacuum collection chamber to the needle. In this example, the vacuum chamber is cylindrical, but may be another shape. In some embodiments, the collection chamber may have a transparent window or the chamber itself may be substantially transparent in order to view the amount of tissue collected and the presence/absence of blood, etc. Dashed lines represent objects or surfaces hidden from view. In one embodiment, negative pressure may be generated in the tissue collection chamber. An attachment from the vacuum collection chamber to a syringe enables negative pressure at the back end of the needle. In some embodiments, the negative pressure is sufficient to provide a suction force that can at least get the tissue sample in the needle. In some embodiments, the operator may have to suction saline to help flush the sample from the needle into the tissue collection chamber. Alternatively, the syringe may be replaced with an external vacuum source via an appropriate attachment.

FIG. 20B shows the sheath with the biopsy needle and resultant coverage of the cutting edge by placement therein. As shown in FIG. 20C, a backward force on the posterior connection (110) causes retraction of the sheath, resulting in exposure of the cutting edge. In one embodiment, the back end of the needle contains an opening for cut tissue to be suctioned into the vacuum collection chamber.

Forward-firing biopsy devices risk surpassing a periphery of the mass or tumor, presenting a danger to patients. Hence, in preferred embodiments, the tip of the needle is placed at the farthest periphery of the mass or tumor, and the biopsy device is made to simultaneously retract and rotate the needle when harvesting tissue/specimen. FIG. 20D is a non-limiting embodiment of an encasement adjacent to the vacuum collection chamber housing the attached syringe/vacuum source, and needle rotating mechanism. A means of drawing back the needle as it rotates, in this case a small spring, is within the encasement. A means for the user to adjust how far the needle withdraws as it rotates and the resultant gross length of harvested tissue, such as a switch attached to the spring, is present. The needle can rotate clockwise or counterclockwise, therefore the cutting edge is positioned in the appropriate direction for cutting tissue when the needle is rotating as it is being drawn back.

One skilled in the art may affix an electric motor that provides for rotational and retractional force upon the needle instead of the spring mechanism. Alternatively, a toothed rod and gear representing one embodiment of a means of generating rotational energy transmitted to the needle via a large spring. In other embodiments, a trigger may be an “on-off” switch for an electric motor where the motor, instead of a spring, provides rotational forces.

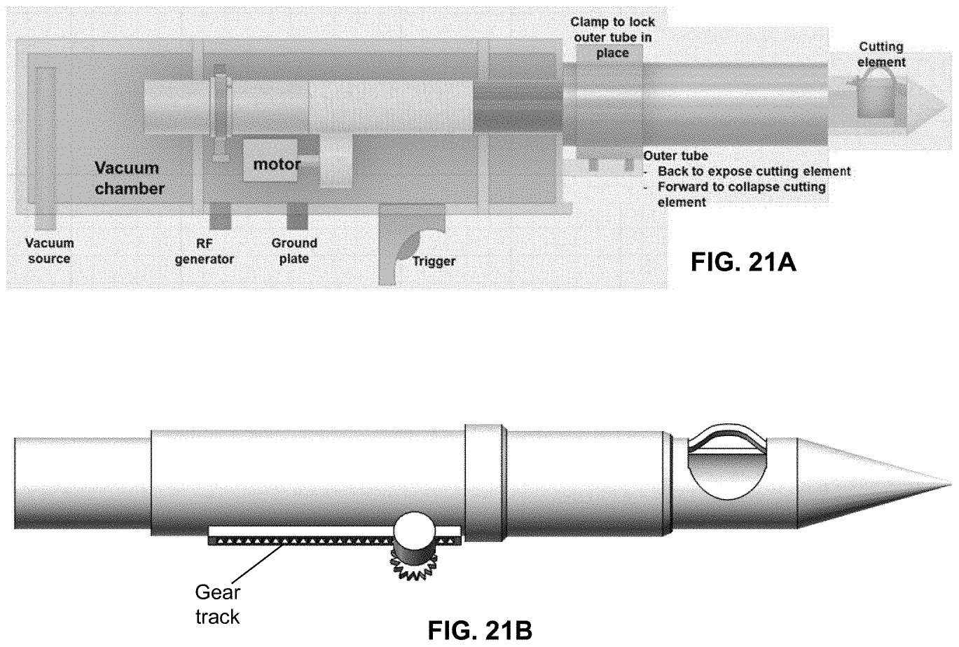

Referring to FIG. 21A, an alternative motor drive may provide a mechanical force needed to move the sheath and rotate the needle, a connection to radiofrequency (RF) electrical source for electrosurgical cutting/coagulation, and a vacuum chamber at the back end of the needle for collecting cut tissue samples. Another alternative mechanism to retract the sheath is shown in FIG. 21B. In some embodiments, the sheath may have a track disposed on its exterior surface. A gear operatively coupled to a motor drive engages the track on the sheath, thereby causing translational movement of the sheath over the needle. In other embodiments, a Luer lock may connect a distal sheath to proximal sheath.

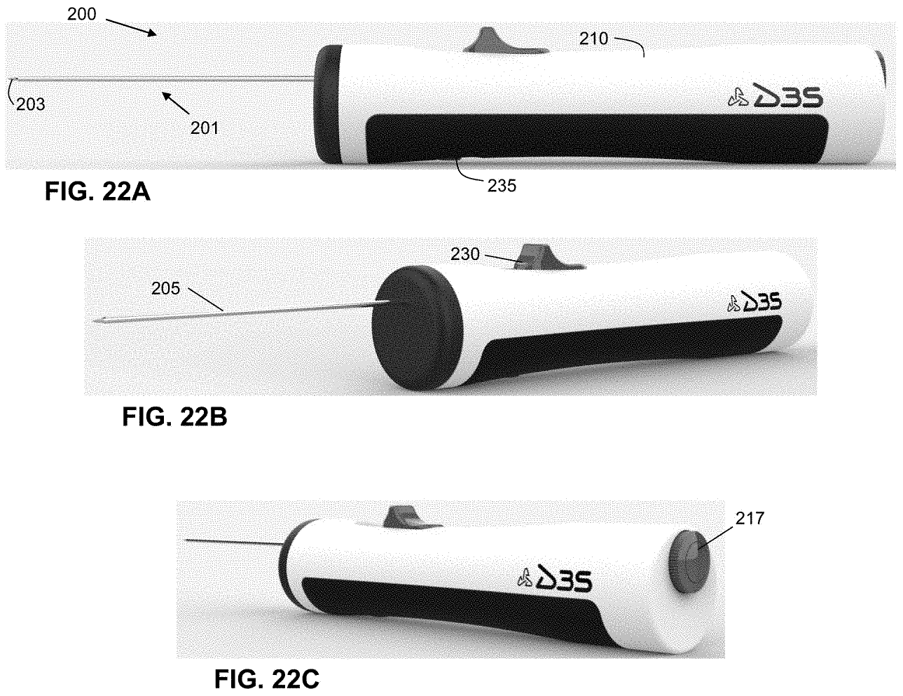

As shown in FIG. 22A-22C, an exemplary embodiment of the biopsy system (200) includes a handle (210) and a biopsy needle (201) extending from the handle. The biopsy needle (201) has a sheath (205) that covers the cutting mechanism disposed at the needle tip (203).

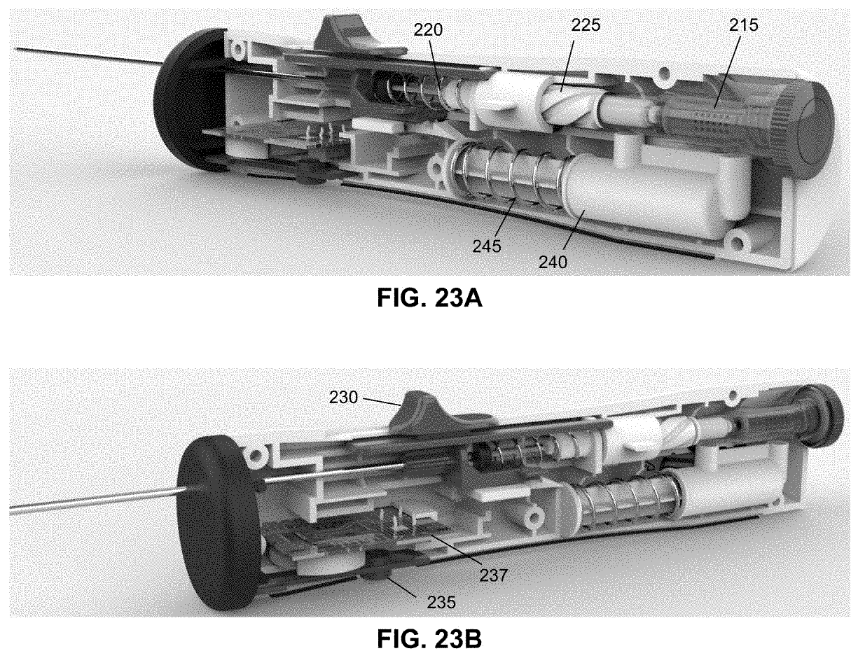

Referring now to FIG. 23A-23B, the internal mechanisms and components of the biopsy system include a trigger (230) that is operatively coupled to a translation spring (220), which is operatively coupled to a rotational screw (225). The needle is fluidly coupled to a vacuum collection chamber (215). The vacuum collection chamber (215) is in connection with an internal syringe (240) and a vacuum spring (245). The vacuum spring (245) is configured to pull the internal syringe (240) that creates negative pressure in the vacuum collection chamber (215). The vacuum collection chamber (215) is coupled to the handle (210) and can be removed from the handle or attached to the handle by threading the cap (217). The electrocoagulation or electrocautery/electrocoagulation activation button (235) and associated electronic components (237), including a power source, are in contact with the inner needle (201) and the outer metal cannula/sheath (205).

### EXAMPLES

The following are non-limiting examples of utilizing the systems of the present invention in a biopsy procedure. It is to be understood that the invention is not limited to the examples that will be described herein. Equivalents or substitutes are within the scope of the invention.

### Example 1

### Bioimpedance Measurements

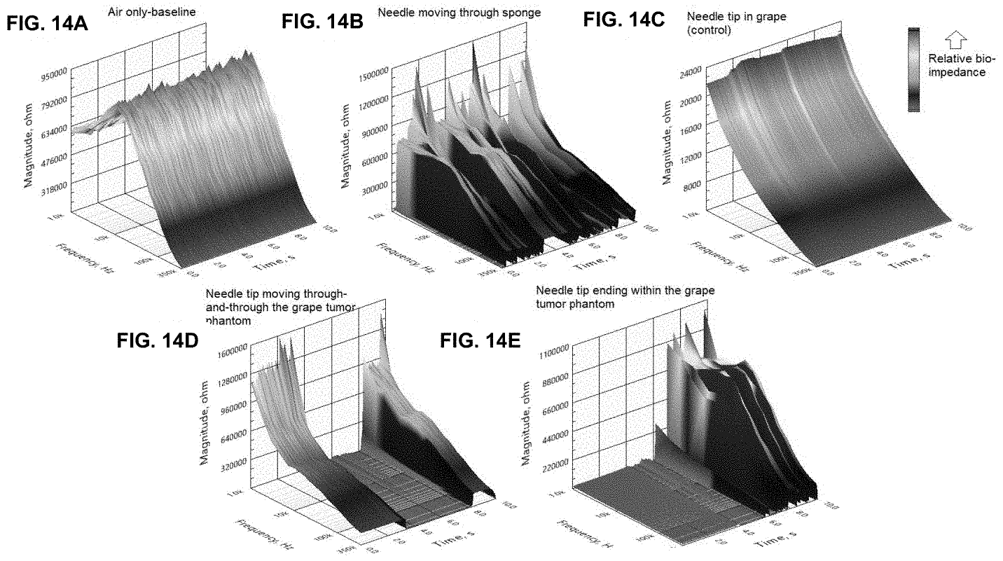

A non-limiting experimental procedure of implementing the present invention is shown in FIG. 13. A multi-purpose sponge (3M) measuring 4.7×3.0×0.6 inches was incised for placement of a tumor phantom and wetted with 15 ml tap water spread evenly throughout the sponge. A green seedless table grape was placed in the incision as a tumor phantom. A Quadra impedance spectrometer (Eliko, Estonia) was connected to a concentric solid coaxial needle electrode (Technomed medical accessories, the Netherlands) with single shunt front-end 2-wire configuration. The electrode needle was then placed in various trajectories corresponding to FIG. 14A-14E.

Impedance magnitude was recorded and displayed in waterfall setting with a signal level set to 100 and measured for 10 seconds at all available default frequencies (in kHz: 1, 2, 3, 7, 11, 17, 23, 31, 41, 61, 89, 127, 179, 251, 349) using Eliko-provided Quadra software (v1.3).

As a control for large relative impedance, impedance of the air was evaluated (FIG. 14A). While placing the electrode needle through the lung tissue phantom (slightly wetted sponge), distinct patches of relatively high impedance (air pockets representing alveoli) and then lower impedance (sponge material with and without water pockets, representing interstitial tissue and/or capillaries) were appreciated (FIG. 14B). As an alternative control, the electrode needle tip was placed in the grape, which demonstrated orders of magnitude less impedance than air across all frequencies (FIG. 14C).

### Example 2

### Biopsy Procedure

As shown in FIG. 10A-10B, the biopsy needle includes a sheath through which the biopsy needle and other accessories can be inserted. The sheath includes integrated electrodes for bioimpedance. Accessories include electrocautery/electrocoagulation device, radiopaque marker insertion device, tissue sealant injector, device to inject filler material, etc.

1. Prepare device: use syringe to apply suction; turn stopcock to preserve vacuum.

2. Referring to FIGS. 11A-11B, measure impedance using the electrodes integrated into the sheath to guide and advance the needle with the outer sheath into patient and position at distal end of tumor.

3. Use mechanics to retract outer sheath to expose expandable curved cutting blade and needle lumen.

4. Use a stopcock to apply vacuum to needle lumen.

5. Rotate device to collect tissue biopsy (manual, motor driven, spring driven) while pulling the device towards the operator.

6. Stop rotation and reposition outer sheath.

7. Collect biopsy using vacuum and store in collection chamber

8. Use stopcock to close vacuum

9. Remove needle unit; sheath (optionally) remains in place

### Example 3

### Biopsy Procedure With Electrocautery/Electrocoagulation Device

1. As shown in FIG. 12, apply electrode pad to patient body and connect to electrocautery/electrocoagulation unit.

2. Connect electrocautery/electrocoagulation unit to biopsy device.

3. Insert electrocautery/electrocoagulation device into sheath and engage using luer adapter.

4. Advance electrocautery/electrocoagulation device tip into biopsy site by measuring bioimpedance using the electrodes integrated into the sheath to guide the needle with the outer sheath into the patient.

5. Push activation button and hold to use electrocautery/electrocoagulation.

6. Rotate needle (mechanical, motor, or spring) while pulling the device towards the operator.

7. Release activation button to inactivate electrocautery/electrocoagulation.

8. Disengage sheath from electrocautery/electrocoagulation unit.

9. Remove electrocautery/electrocoagulation unit.

### Example 4

### Biopsy Procedure Triopsy Edge Complex Device

1. Again, referring to FIG. 12, apply grounding pad to the patient and attach to electrocautery/electrocoagulation unit. Attach electrocautery/electrocoagulation unit to biopsy device.

2. Insert needle and sheath into patient using bioimpedance guided visualization.

3. Position within the tumor, preferably distally for pullback biopsy.

4. Press start button to activate device. Solid green ready light appears.

5. Press start button to begin biopsy process. Green light begins to blink.

6. Outer sheath retracts.

7. Vacuum starts.

8. Electrosurgery/electrocoagulation signal directed to cutting blade.

9. Needle begins to rotate for n rotations (n=1-20).

10. Electrosurgery/electrocoagulation inactivated.

11. Outer sheath extends distally.

12. Vacuum continues to collect tissue.

13. Vacuum turns off.

14. Solid green light reappears—ready light.

15. Reposition biopsy device to starting position.

16. Depress and hold button to activate electrocautery/electrocoagulation or electrocoagulation.

17. Inactivate biopsy parts.

18. Withdraw outer sheath to expose electrocautery/electrocoagulation or electrocoagulation.

19. Blinking red light is activated, green light off.

20. Physician moves needle/sheath outward to cauterize biopsy tract.

21. Release button to stop electrocautery/electrocoagulation unit.

22. Blinking red light is inactivated.

23. Outer sheath advanced distally.

24. Electrocautery/electrocoagulation or electrocoagulation unit shuts down.

### Example 5

### Biopsy Procedure

1. Referring to FIGS. 23A-23B, cock/load the trigger (230) in direction of the needle. This compresses the translation spring (220) and creates negative pressure in the vacuum chamber (215) while causing the sheath (205) to move forward, covering the expandable cutting blade on the inner needle (201).

2. Insert the needle (201) and sheath (205) into the patient.

3. Position the needle tip (203) in the tumor, preferably distally for pullback biopsy.

4. Activate the trigger (230). This causes the sheath (205) to retract, the cutting blade near the tip of the inner needle to expand, and negative pressure in the vacuum chamber (215) to suction a small portion of the tissue to be biopsied into the tip of the inner rotating and cutting needle. The inner cutting needle (201) simultaneously rotates while being retracted along the axis of the rotational screw (225) while suctioning the cut tissue. The inner cutting needle (203) eventually completes its rotations and retracts into the outer sheath (205) which clips the tail end of the coil of harvested tissue that has been suctioned into the device.

Various modifications of the invention, in addition to those described herein, will be apparent to those skilled in the art from the foregoing description. Such modifications are also intended to fall within the scope of the appended claims. Each reference cited in the present application is incorporated herein by reference in its entirety.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. Reference numbers recited in the below claims are solely for ease of examination of this patent application, and are exemplary, and are not intended in any way to limit the scope of the claims to the particular features having the corresponding reference numbers in the drawing. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting of”, and as such the written description requirement for claiming one or more embodiments of the present invention using the phrase “consisting of” is met.

What is claimed is:

1. A bioimpedance system (100) for guided positioning of an instrument inside a body (105), said system (100) comprising:
a. the instrument (110) for insertion into the body (105);
b. one or more electrical connections (164) operatively coupled to the instrument;
c. an impedance spectrometer (120) operatively coupled to the one or more electrical connections (164), wherein the impedance spectrometer (120) is configured to pass electrical current to the one or more electrical connections (164); and
d. a processor (130) operatively coupled to the impedance spectrometer, wherein the processor (130) is configured to execute computer-readable instructions that cause the processor (130) to obtain bioimpedance measurements from the one or more electrical connections (164), whereby a local environment surrounding the instrument is determined (110) from the bioimpedance measurements, thereby providing guidance during insertion and positioning of the instrument (110) within the body (105).

2. The system (100) of claim 1, wherein the electrical connections (164) comprise electrodes.

3. The system (100) of claim 2, wherein the electrodes comprise conductive strips, ribbons, or wires disposed along the surface of the instrument, or embedded or disposed within the instrument.

4. The system (100) of claim 2, wherein the electrodes comprise multiple concentric telescoping tubes each with an electrically active exposed tip or surface.

5. The system (100) of claim 1, wherein the instrument (110) is comprised of conductive and insulated portions, wherein the conductive portions function as additional electrical connections.

6. The system (100) of claim 1, wherein the instrument (110) is a needle, a catheter, probe, clamp, forceps, or surgical tool.

7. The system (100) of claim 6, wherein the instrument (110) is a radiofrequency ablation (RFA) needle, a cryoablation needle, or a microwave ablation needle.

8. The system (100) of claim 6, wherein the instrument (110) is catheter, wherein a plurality of guide-wire electrodes are disposed lengthwise inside the catheter.

9. The system (100) of claim 1, wherein the instrument (110) is a biopsy needle having a needle sheath, wherein the electrical connections comprise electrodes disposed on a surface of the needle sheath.

10. The system (100) of claim 1, wherein the instrument (110) is an electrosurgical tool, wherein the electrosurgical tool is operatively coupled to an electrosurgical pulse generator.

11. The system (100) of claim 10, wherein the electrosurgical tool is a scalpel or knife.

12. The system (100) of claim 1, wherein the instrument (110) is further configured for cauterization.

13. The system (100) of claim 1, wherein the processor (130) is operatively coupled to a display (140), which displays electrical settings, impedance data, and/or an impedance map.

14. A method of guiding a position of an instrument (110) inside a body (105), comprising:
a. providing the bio-impedance guided system (100) of claim 1;
b. inserting the instrument (110) into the body (105);
c. obtaining bio-impedance measurements using the one or more electrical connections (164); and
d. determining a position or direction of the instrument (110) based on the multiple bioimpedance measurements, wherein the bioimpedance measurements indicate bone, tissue type, and/or fluids in a local environment surrounding the instrument (110).

15. The method of claim 14, further comprising advancing the instrument (110) inside the body, changing direction of movement of the instrument, retracting the instrument, and/or ceasing movement of the instrument based on the bioimpedance measurements.

16. A biopsy system (200) for harvesting a target tissue, comprising:
a. a needle having a tip for insertion into tissue, wherein a lumen is disposed in the needle;
b. an aperture disposed at or near the tip of the needle, said aperture fluidly connected to the lumen;
c. a cutting mechanism adapted to cut tissue, said cutting mechanism having at least a portion thereof disposed in or over the aperture;
d. a sheath slidably disposed around an exterior surface of the needle, the sheath adapted to move between at least an open position where the aperture and cutting mechanism are exposed, and a closed position where the aperture and cutting mechanism are covered by the sheath;
e. a mechanism for cauterizing tissue that contacts said cauterizing mechanism;
f. a mechanism for rotating the needle;
g. one or more electrical connections operatively coupled to the needle;
h. an impedance spectrometer (120) operatively coupled to the one or more electrical connections, wherein the impedance spectrometer is configured to pass electrical current to the one or more electrical connections;
i. a processor (130) operatively coupled to the impedance spectrometer, wherein the processor (130) is configured to execute computer-readable instructions that cause the processor (130) to perform operations comprising:
i. obtaining bioimpedance measurements from the one or more electrical connections; and
ii. determining a composition of a local environment surrounding the needle, thereby providing guidance during insertion and positioning of the needle within the body;
ps wherein when the needle is inserted into a body, bio-impedance measurements are used to guide and position the needle at the target tissue, wherein the needle tip is placed at a periphery of the target tissue, the sheath is deployed in the open position, the needle is rotated via the rotation mechanism and simultaneously retracted from the periphery, and the cutting mechanism cuts the tissue and directs said cut tissue into the aperture and further into the lumen, while contacting tissue is cauterized by the cauterizing mechanism.

17. The system (200) of claim 16, wherein the cutting mechanism comprises a dome-shape structure having a leading cutting edge projecting from the needle.

18. The system (200) of claim 16, wherein the cutting mechanism is deployable, wherein the deployable cutting mechanism comprises one of the following:
i. at least one cylindrical or filament wire or an expandable dome-shape structure;
ii. at least one flat wire with a first side and a second side, wherein the first side is for cutting and the second side is for cauterization or coagulation; or
iii. a nitinol memory wire that is pre-configured to assume a desired conformation.

19. The system (200) of claim 16, wherein the electrical connections are configured to apply electrical current for cauterizing tissue, coagulating blood, obtaining the multiple bioimpedance measurements to guide needle insertion and positioning, and/or initiating electron-dependent biochemical processes.

20. The system (200) of claim 16, wherein the processor (130) is operatively coupled to a display (140), which displays electrical settings, impedance data, and/or an impedance map.
